(19) United States
(12) Patent Application Publication
(10) Pub. No.: US 2018/0327461 A1
(43) Pub. Date: Nov. 15, 2018

(54) COMPOSITIONS AND METHODS FOR CONTROL OF VECTOR-BORNE DISEASE

(71) Applicants: Ravi DURVASULA, Albuquerque, NM (US); ARORA Arinder Humar, Albuquerque, NM (US); STC.UNM, Albuquerque, NM (US)

(72) Inventors: Ravi Durvasula, Albuquerque, NM (US); Arinder Kumar Arora, Albuquerque, NM (US)

(21) Appl. No.: 15/574,679

(22) PCT Filed: May 18, 2016
(86) PCT No.: PCT/US2016/033029

§ 371 Date: Nov. 16, 2017

Related U.S. Application Data
(60) Provisional application No. 62/264,434, filed on Dec. 8, 2015.
(60) Provisional application No. 62/163,087, filed on May 18, 2015.

(51) Int. Cl. C07K 14/435; C07K 14/47; A01N 63/00; C07K 16/12

(52) U.S. Cl. CPC C07K 14/43572; C07K 14/4723; C07K 2319/30; C07K 16/1203; C07K 16/1228; A01N 63/00

(57) ABSTRACT

This disclosure describes a genetically-modified microbe that is a symbiont of an animal that is a vector organism for a pathogenic microbe, a paratransgenic organism that includes the genetically-modified microbe, and methods involving use of the genetically-modified microbe and/or the paratransgenic organism. Generally, the genetically-modified microbe includes a heterologous polynucleotide that encodes a heterologous polypeptide that reduces transmission of the pathogenic microbe.

### CROSS-REFERENCE TO RELATED APPLICATION

This application claims priority to U.S. Provisional Patent Application No. 62/163,087, filed May 18, 2015, and U.S. Provisional Patent Application No. 62/264,434, filed Dec. 8, 2015, each of which is incorporated herein by reference.

### SUMMARY

This disclosure describes, in one aspect, a genetically-modified microbe that is a symbiont of an animal that is a vector organism for a pathogenic microbe. Generally, the genetically-modified microbe includes a heterologous polynucleotide that encodes a heterologous polypeptide that reduces transmission of the pathogenic microbe.

In some embodiments, the genetically-modified microbe is, or is derived from, Pantoea agglomerans.

In some embodiments, the heterologous polypeptide includes an antimicrobial peptide. In some of these embodiments, the antimicrobial peptide includes melittin or scorpine-like molecule (SLM).

In some embodiments, the heterologous polypeptide can include an antibody that specifically binds to the pathogenic microbe or an antibody fragment that specifically binds to the pathogenic microbe.

In some embodiments, the heterologous polypeptide can include an effective portion of an antimicrobial peptide fused to at least a fragment of an antibody that specifically binds to the pathogenic microbe.

In another aspect, this disclosure describes a paratransgenic organism that includes any embodiment of the genetically-modified microbe summarized above.

In another aspect, this disclosure describes a method of reducing transmission of a pathogen between members of a population of host organisms. Generally, the method includes applying a composition that includes any embodiment of the genetically-modified microbe summarized above to a population of host organisms; and allowing vector organisms carrying or at risk of carrying the pathogen to acquire the composition comprising the genetically-modified microbe.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. (A) SLM sequence (SEQ ID NO: 1) showing predicted domains with helix-coil structure. (B) 3-D structure of SLM as depicted by I-TASSER.

FIG. 2. Both melittin and SLM were to be more toxic to X. fastidiosa than P. agglomerans. 10⁵-10⁶ CFUs of P. agglomerans and X. fastidiosa were treated with AMPs. OD₆₀₀ was measured 24 hours after treatment of P. agglomerans as it grows at a fast rate; while X. fastidiosa grows at a slow rate and it was cultured 24 hours after treatment with an anti-microbial peptide (AMP) and CFUs were counted. P. agglomerans OD₆₀₀ after treatment with (A) melittin, (C) SLM; X. fastidiosa CFUs counts after treating with (B) melittin, (D) SLM.

FIG. 3. (A) Schematic diagram depicting pore formed by HlyB and HlyD in combination with TolC in the membrane of Gram negative bacteria and a protein with HlyA secretion signal passing through it. (B) Schematic description of introducing effector molecules at 5′ end of E-tag, which was in frame with HlyA secretion signal.

FIG. 4. (A) Western blot showing secretion and accumulation of melittin and SLM conjugated to HlyA secretion signal by transformed P. agglomerans lines in spent media. Spent media from transformed P. agglomerans lines were concentrated using Micron 10 kDa filters. Concentrated spent medium was tested using an anti-E-tag antibody. Lane 1: ladder; lane 2: Wild type P. agglomerans; lane 3: HlyA secretion signal only; lane 4: melittin conjugated to HlyA secretion signal; lane 5: SLM conjugated to HlyA secretion signal. (B) and (C). Western blots showing secretion and accumulation of melittin and SLM conjugated to HlyA secretion signal by transformed P. agglomerans lines in the sharpshooter gut. Extracts from homogenized sharpshooters were tested for presence of AMPs using an E-tag antibody. (B) Lane 1: ladder; lane 2: sharpshooter fed on P. agglomerans expressing melittin conjugated to HlyA secretion signal; lane 3: sharpshooter fed on wild type P. agglomerans (C) Lane 1: ladder; lane 2: sharpshooter fed on P. agglomerans expressing SLM; lane 3: sharpshooter fed on wild type P. agglomerans.

FIG. 5. Paratransgenic sharpshooters, which acquired anti-microbial peptide (AMP)-producing P. agglomerans were refractory to X. fastidiosa acquisition. P. agglomerans was painted on grape stems after mixing with guar gum. The sharpshooters were allowed to feed on these plants for 48 hours before putting them in cage having X. fastidiosa infected plant in it for 48 hours. After X. fastidiosa acquisition the sharpshooters were collected and two sharpshooters was kept on each naive grape plant for 24 hours. These sharpshooters were surface sterilized and X. fastidiosa presence was tested using rt-PCR. (A) X. fastidiosa CFUs per insect head. (B) Prevalence of X. fastidiosa in sharpshooter heads. p values: a, p=0; b, p=0; c, p=0.0048; d, p=0.1388; e, p=0; f, p=0.0003; g, p=0.946; h, p=0.0098; i, p=0.0319; j, p=0.8270.

FIG. 6. Paratransgenic sharpshooters, which acquired anti-microbial peptide (AMP)-producing P. agglomerans did not transmit X. fastidiosa to naive grape plants. P. agglomerans were painted on grape stems after mixing with guar gum. The sharpshooters were allowed to acquire P. agglomerans from P. agglomerans painted plants for 48 hours before acquisition access of 48 hours on X. fastidiosa infected grape plants. These sharpshooters were collected and two sharpshooters were then kept on each naive grape plant for 24 hours. These plants were kept for 30 weeks in greenhouse before testing them for presence of X. fastidiosa using rt-PCR. (A) Percent plants infected with X. fastidiosa. (B) Regression line using percent transmission efficiency as dependent and percent acquisition as independent variable.

FIG. 7. Confirmation of secretion and accumulation of melittin conjugated to HlyA secretion signal by transformed P. agglomerans lines in spent media as well as withing sharpshooter gut. (A) Spent media were concentrated as described before and was analyzed using anti-melittin bleed via Western blot. lane 1: melittin conjugated to HlyA secretion signal; lane 2: synthetic melittin; lane 3: ladder. (B) Solution from sharpshooter was prepared as described earlier. This solution was analyzed using anti-melittin bleed. Lane 1: ladder; lane 2: sharpshooter fed on P. agglomerans expressing melittin conjugated to HlyA secretion signal; lane 3: sharpshooter fed on wild type P. agglomerans.

FIG. 8. Cloning of melittin coding region or SLM coding region into pEHLYS2-SD

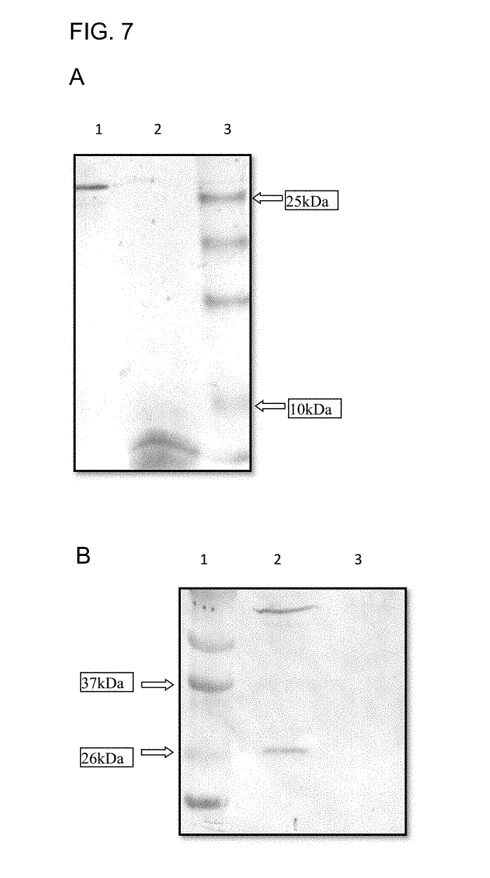

FIG. 9. Plasmid pVDL9.3.

### DETAILED DESCRIPTION OF ILLUSTRATIVE EMBODIMENTS

Vector-borne diseases can afflict human health and/or cause diseases in animals and agriculture crops. Pierce's disease of grapevine is a disease that can kill grape plants and is a threat to grape production in the United States. Pierce's disease is caused by a rod shaped, Gram negative, xylem-limited bacterium, Xylella fastidiosa. Leafhoppers commonly known as sharpshooters are vectors of X. fastidiosa. Homalodisca vitripennis, the glassy-winged sharpshooter (GWSS), is a common vector of X. fastidiosa due, at least in part, to its long range mobility and high fecundity. Similar to other vector-borne diseases such as, for example, malaria and Chagas disease, the spread of Pierce's disease is often checked by controlling vector population using insecticides. Insecticide use is associated with concerns such as, for example, resistance development, environment pollution, and/or resurgence.

In contrast, this disclosure describes insecticide-free biological strategies for control of Pierce's Disease. One strategy involves paratransgenic control of Pierce's disease. Paratransgenic control relies on a vector symbiont to deliver anti-pathogen molecules inside the vector body to make the vector incompetent to carry the pathogen. The incompetent vector fails to transmit the pathogen, hence breaking the disease cycle. In this context, this disclosure describes paratransgenic control for Pierce's disease and for the first time shows a decrease in X. fastidiosa transmission by paratransgenic GWSS using recombinant symbiotic bacterium, Pantoea agglomerans. P. agglomerans is a grape endophyte, which was selected to deliver anti-X. fastidiosa molecules inside the foregut of GWSS.

Another strategy involves a directly delivering an antimicrobial peptide (AMP) to a surface of a crop plant so that the AMP compound is accessible for acquisition by the vector organism that carries the pathogen. Acquisition of the AMP compound by the vector organism can decrease the likelihood and/or extent to which the vector transmits the pathogen among a population of crop plants.

Paratransgenic models previously developed that exhibited a decrease in pathogen carriage inside the insect gut failed to exhibit a decrease in pathogen transmission (Durvasula et al., 1997, Proc. Natl. Acad. Sci. 94:3274-3278; Wang et al., 2012, Proc. Natl. Acad. Sci. 109:12734-12739). This is also the first time that paratransgenic control has been used to control a vector-borne disease in agriculture setting.

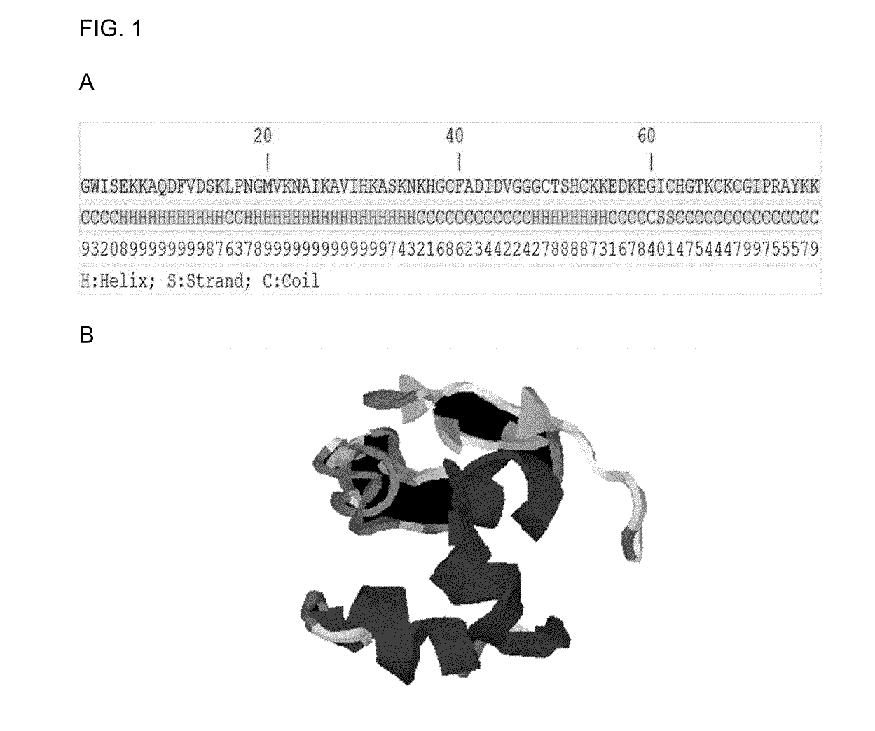

Antimicrobial peptides (AMPs) are defense molecules synthesized by higher eukaryotes against microbes and parasites. These molecules can act against different pathogens through various mechanisms including, for example, making pores in the membrane, interfering with protein or nucleic acid synthesis, and/or interfering with different signal transduction pathways. Melittin, a 26 amino acid peptide having an alpha-helix structure, has been isolated from honey bee venom. Melittin can kill cells by making pores in the cell membrane or by inducing apoptosis. A second anti-microbial peptide selected to block X. fastidiosa transmission is scorpine-like molecule (SLM). SLM has been isolated from the venom of the scorpion Vaejovis mexicanus. It is a 77 amino acid peptide and has 44% homology with scorpine, another anti-microbial peptide from scorpion Pandinus imperator. I-TASSER software analysis (Roy et al., 2010, Nature Protocols 5(4):725-738; Yang et al., 2015, Nature Methods 12(1):7-8) reveals SLM to have three coil-helix structures (FIG. 1).

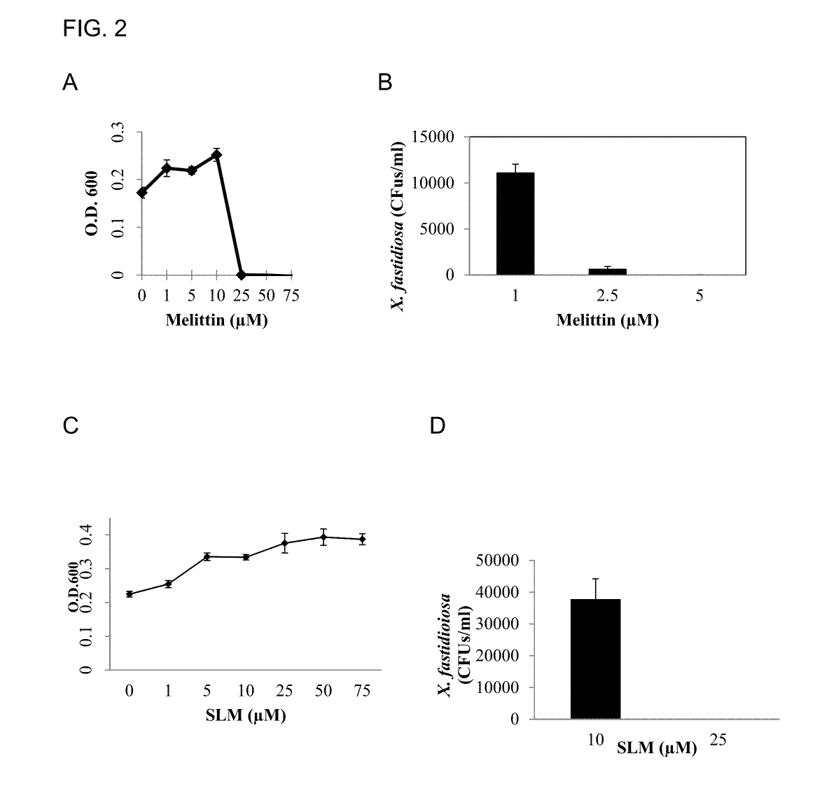

The activity of each peptide was tested against X. fastidiosa and P. agglomerans. In vitro studies have shown that melittin killed P. agglomerans at a concentration of 25 μM, which was five times higher than melittin concentration that killed X. fastidiosa (5 μM) (FIGS. 2A and 2B). Similarly, SLM killed X. fastidiosa at a concentration of 25 μM, but did not kill P. agglomerans at the highest SLM concentration tested (75 μM) (FIGS. 2C and 2D). The ability of P. agglomerans to withstand higher concentrations of AMPs means it can be transformed to produce melittin and SLM. These transformed strains can be used to produce paratransgenic sharpshooters to inhibit X. fastidiosa transmission.

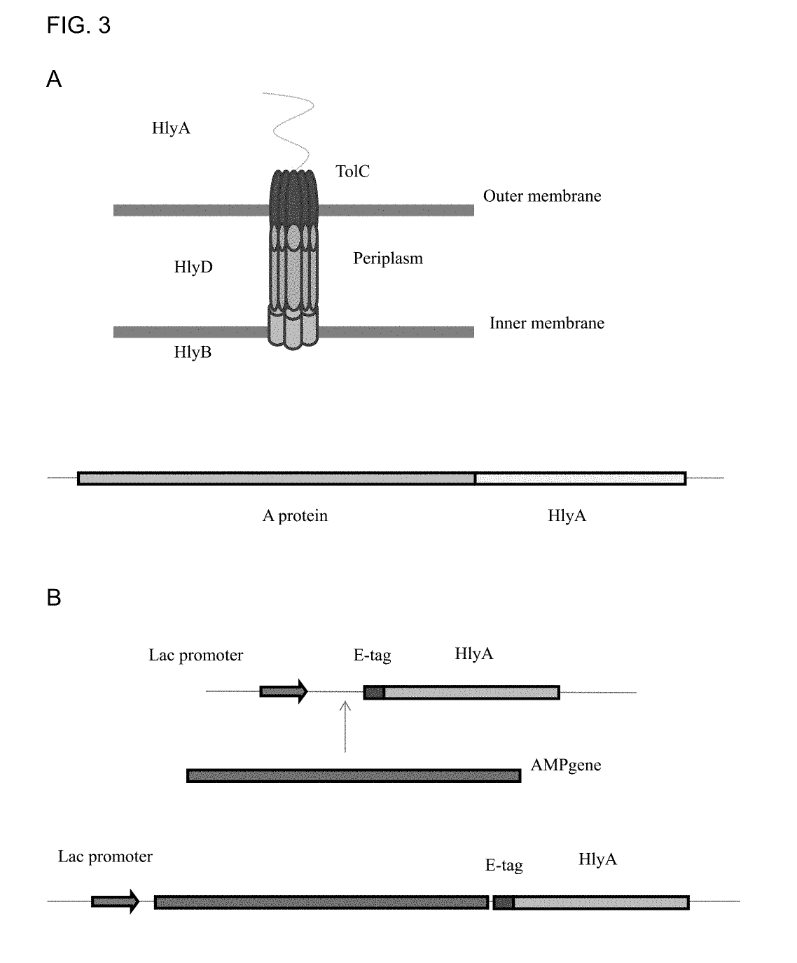

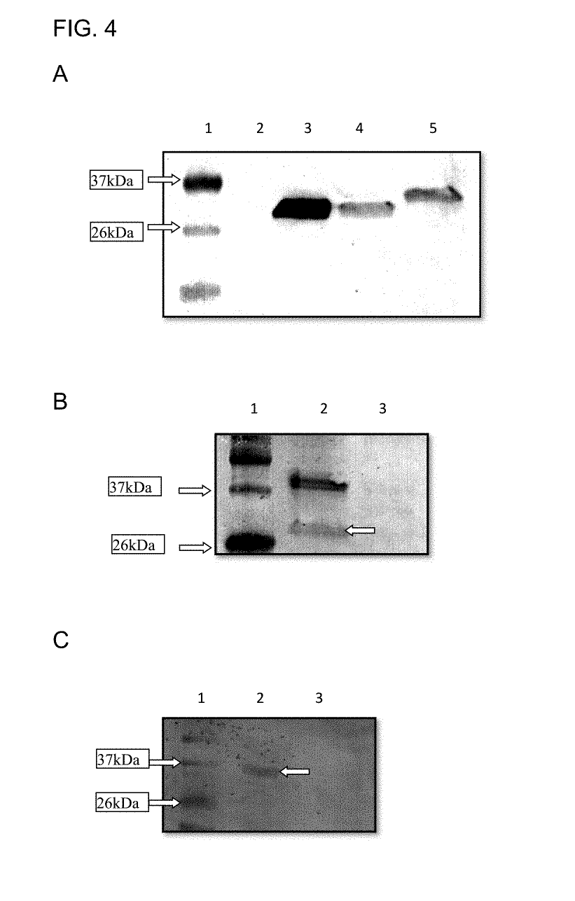

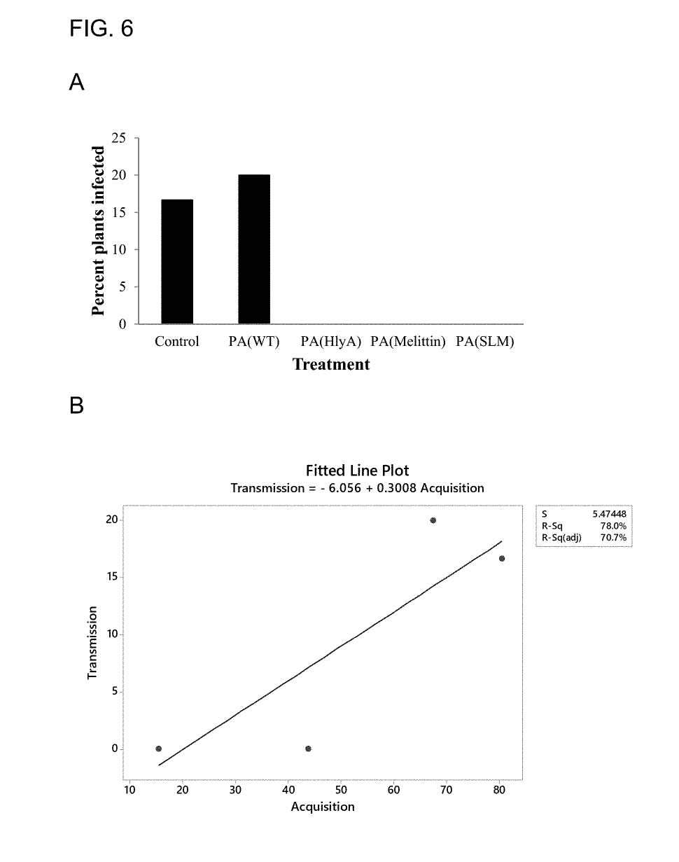

The E. coli hemolysin secretion system, which has been used to secrete active molecules into the extracellular environment, was employed to generate P. agglomerans strains that can secrete melittin and SLM. E. coli hemolysin secretion system is composed of pore forming proteins HlyB and HlyD and a carboxyl terminal HlyA secretion signal. Pores formed by HlyB and HlyD in combination with pores formed by TolC provide proteins with an HlyA secretion signal passage to the outside environment (FIG. 3A). A coding region encoding either melittin or SLM was introduced into plasmid pEHLYA2-SD at the 5′ end of an E-tag, which was in frame with the HlyA secretion signal (FIG. 3B). P. agglomerans was transformed with pEHLYA2-SD and pVDL9.3, a plasmid with HlyB and HlyD coding regions. These strains secreted melittin and SLM with the HlyA secretion signal intact (FIG. 4A). An anti-E-tag antibody was used to detect secretion of melittin and SLM in spent media. Melittin secretion was re-confirmed using anti-melittin serum (FIG. 7A).

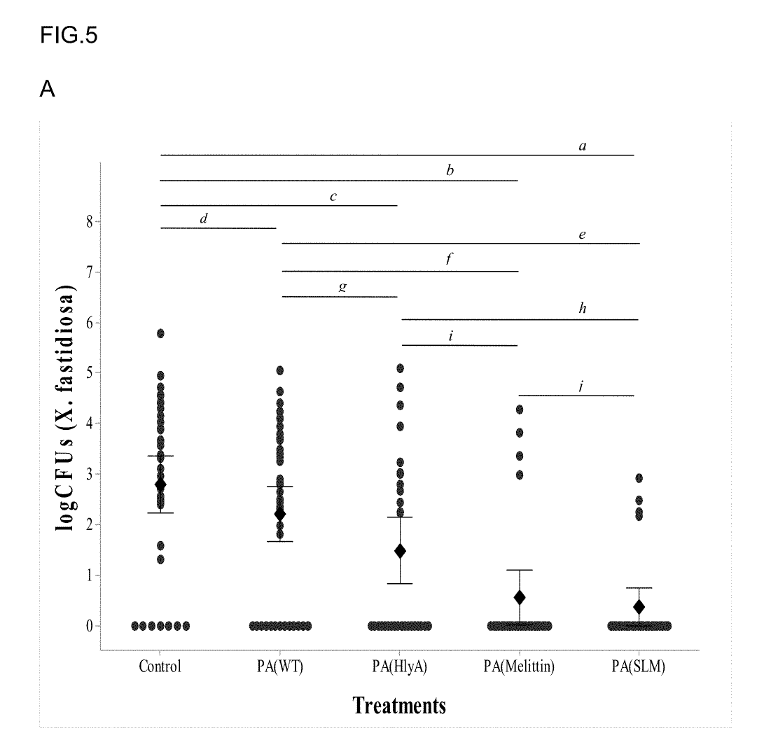

P. agglomerans strains (10¹⁰ CFUs) were mixed with guar gum and painted on to grapevine stems. GWSS were allowed to feed on these plants for 48 hours. After P. agglomerans acquisition, the sharpshooters were allowed to acquire X. fastidiosa from infected grape plants for 48 hours. These sharpshooters were collected and then two sharpshooters were given inoculation access of 24 hours on a naive grape plant. The sharpshooters that were fed AMP-producing P. agglomerans prior to X. fastidiosa acquisition exhibited reduced X. fastidiosa acquisition. The sharpshooters that were fed melittin-secreting or SLM-secreting P. agglomerans carried, on average, only 4.28% and 0.22%, respectively, of the X. fastidiosa CFUs carried by the control (p<0.00001) (FIG. 5A, a (PA(SLM)) and b (PA/(Melittin))). Moreover, the number of paratransgenic GWSS carrying X. fastidiosa in their foregut also decreased significantly. 80.55% of control sharpshooters acquired X. fastidiosa, while only 15.38% of GWSS harboring melittin-secreting or SLM-secreting P. agglomerans were found to carry X. fastidiosa in their foregut (p<0.00001) (FIG. 5B).

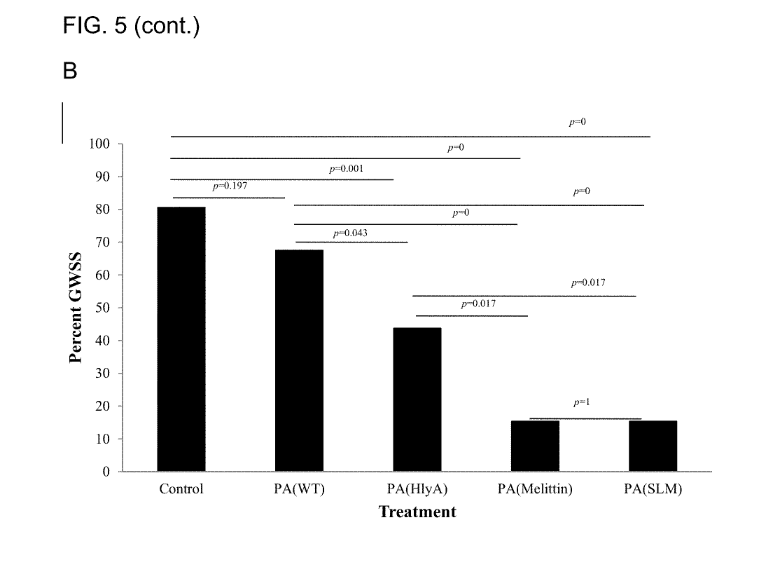

The decrease in X. fastidiosa acquisition by H. vitripennis translates into a decrease in transmission. Paratransgenic GWSSs, which acquired melittin-producing or SLM-producing P. agglomerans strains prior to acquisition of X. fastidiosa, failed to transmit X. fastidiosa to the naive plants (FIG. 6A, PA(Melittin) and PA(SLM)). In contrast, control sharpshooters and sharpshooters carrying wild-type P. agglomerans transmitted X. fastidiosa to 16.67% and 20% of test plants, respectively (FIG. 6A, Control and PA(WT)).

The sharpshooters were tested for the presence of anti-microbial peptide molecules to confirm that the observed decrease in transmission was a result of anti-microbial peptide activity in sharpshooter gut. Plants were inoculated with 10¹⁰ CFUs of P. agglomerans per plant via guar gum. GWSS were allowed to feed on these plants for 48 hours. After 48 hours, these sharpshooters were removed from plants, surface sterilized, and tested for accumulation of peptides. Western blot analysis confirmed presence of AMPS inside the sharpshooter body (FIGS. 4B and 4C), detecting protein a band at approximately 29 kDa for melittin and a protein band at approximately 31 kDa for SLM, which was absent in control insects. Further, the presence of melittin was confirmed using anti-melittin serum, which did not cross-react with any of the sharpshooter proteins (FIG. 7B).

Previous paratransgenic models have shown a decrease in pathogen/parasite acquisition by the vectors, but none of them has shown a decrease in disease transmission (Durvasula et al. 1997, Wang et al. 2012). This is the first report demonstrating a decrease in pathogen transmission by paratransgenic sharpshooters, which were refractory to X. fastidiosa acquisition.

The spread of Pierce's disease and other vector-borne diseases is conventionally kept in check by controlling vector populations with insecticides. Insect vectors can, however, develop resistance against various insecticides. Paratransgenic control of these diseases is an alternative that can be employed in the field to decrease transmission. It can also be included in integrated vector management. Paratransgenic control can reduce disease spread and also can decrease reliance on chemical pesticides. No adverse physiological effects (e.g., decreased feeding or early mortality) were observed in the sharpshooters carrying P. agglomerans strains. This indicates that paratransgenic sharpshooters should be able to complete their life cycle and there will not be any selection pressure on these insects, which is the main cause of resistance development.

The sharpshooter foregut, as compared to the grape plants, carries far fewer X. fastidiosa CFUs, which makes the insect gut an attractive place to attack X. fastidiosa. AMPs expressed by P. agglomerans encounter very few bacteria within sharpshooter gut, exerting less selection pressure on the bacteria. Further, a low quantity of active molecules can combat the pathogen within the gut. These are all factors that inhibit development of resistance. Moreover, single chain fragment variables (scFvs) specific to a X. fastidiosa surface protein, mopB, can be expressed in tandem with active AMPs or as antibody:AMP chimeras to increase the efficacy. The tandem expression of antibodies can further inhibit development of resistance.

In some embodiments, calcium-alginate microparticles can be used to disseminate the genetically-modified bacteria in the field. These microparticles not only provide a physical barrier between the bacteria and the outer environment to decrease the environmental contamination, but also can result in tolerance against desiccation and UV radiations. Microparticles containing recombinant P. agglomerans may be applied to fields in, for example, late spring for the newly emerging sharpshooters to acquire AMP-expressing bacteria. This will make the sharpshooter incompetent of transmitting the pathogen, thereby facilitating transmission blockage.

While described herein in the context of an exemplary embodiment in which the vector animal is the glassy-winged sharpshooter, H. vitrpennis, the genetically-modified symbiont can be designed for use with, and the methods described herein may be practiced using, any suitable vector animal. For example, whitefly, aphids, leafhoppers, and thrips transmit deadly diseases to crop plants ranging from cotton to papaya to rice. These insects carry different symbionts that enhance their fitness and also provide resistance against biotic and abiotic stresses. These symbionts may be exploited as a Trojan Horse to inhibit transmission of pathogens that are transmitted by these insects. Exemplary alternative vector species include those listed in Table 1.

Also, while described herein in the context of an exemplary embodiment in which the host plant is grapevine, citrus, or olive, the methods described herein may be used to control a pathogen of any suitable plant. For example, X. fastidiosa can be a pathogen of plants including, but not limited to, grapevines (e.g., Vitis spp. such as V. vinifera, V. labrusca, V. riparia, V. aestivalis, etc., and/or hybrids of two or more Vitis spp.), citrus, (e.g., Citrus spp. such as C. medica, C. maxima, C. reticulata, C. micrantha, C. limettioides, C. limetta, C. aurantium, etc., and/or hybrids of two or more Citrus spp.), olive (e.g., Olea spp. such as O. europaea, O. sylvestris, etc., and/or hybrids of two or more Olea spp.), mulberry (e.g., Morus spp. such as M. alba, M. nigra, M. rubra, M. celtidifolia, etc. and/or hybrids of two or more Morus spp.), oleander (e.g., Nerium oleander), periwinkle (e.g., Catharanthus roseus), ragweed (e.g., Ambrosia spp. such as A. artemisiifolia, A. trifida, etc., and/or hybrids of two or more Ambrosia spp.), plum (e.g., Prunus spp. such as P. domestica, P. salicina, P. nigra, P. armeniaca, etc., and/or hybrids of two or more Prunus spp.), sycamore (e.g., Planatus spp. such as P. occidentalis, P. orientalis, P. racemose, etc., and/or hybrids of two or more Planatus spp.), tobacco (e.g., Nicotiana spp. such as N. tabacum etc. and/or hybrids of two or more Nicotiana spp.), clover (e.g., Trifolium spp., including white clover (T. repens), red clover (T. pratense), crimson clover (T. incarnatum), etc., and/or hybrids of two or more Trifolium spp.), lilac (e.g., Syringa spp. such as S. vulgaris etc. and/or hybrids of two or more Syringa spp.), snowberry (e.g., Symphoricarpos spp. such as S. albus, S. oreophilus, etc., and/or hybrids of two or more Symphoricarpos spp.), elderberry (e.g., Sambucus spp., including blue elderberry (S. cerulea), American elder (S. canadensis), etc., and/or hybrids of two or more Sambucus spp.), blackberry (e.g., Rubus spp., including R. laciniatus, California blackberry (R. ursinus), etc., and/or hybrids of two or more Rubus spp.), mugwort (e.g., Artemisia spp. such as A. vulgaris etc. and/or hybrids of two or more Artemisia spp.), elm (e.g., Ulmus spp. such as American elm (U. americana), European white elm (U. laevis), etc. and/or hybrids of two or more Ulmus spp.), goldenrod (e.g., Solidago spp. such as S. bicolor, S. canadensis, etc. and/or hybrids of two or more Soldago spp.), and/or oak (e.g., Quercus spp. such as white oak (e.g., Q. alba), northern red oak (e.g., Q. rubra), etc., and/or hybrids of two or more Quercus spp.).

Thus, while described herein in the context of an exemplary embodiment in which the disease being treated in Pierce's Disease of grapevine, the methods described herein may be used to treat any suitable disease such as, for example citrus variegated chlorosis (Table 1), coffee leaf scorch, almond leaf scorch, oleander leaf scorch, phony peach, alfalfa dwarf, or olive quick decline syndrome.

TABLE 1

| Plant | Disease | Pathogen | Vector | Symbiont |
|---|---|---|---|---|
| Grapevine | Pierce's disease | X. fastidiosa | H. vitripennis | P. agglomerans |
| Grapevine | Pierce's disease | X. fastidiosa | Graphocephala | P. agglomerans |
| | | | atropunctata | |
| Grapevine | Pierce's disease | X. fastidiosa | Draeculacephala | P. agglomerans |
| | | | minerva | |
| Grapevine | Pierce's disease | X. fastidiosa | Xyphon | P. agglomerans |
| | | | (Carneocephala) | |
| | | | fulgida | |
| Grapevine | Pierce's disease | X. fastidiosa | Homalodisca | P. agglomerans |
| | | | liturata | |
| Grapevine | Pierce's disease | X. fastidiosa | Homalodisca | P. agglomerans |
| | | | insolita | |
| Grapevine | Pierce's disease | X. fastidiosa | Kolla paulula | P. agglomerans |
| Grapevine | Pierce's disease | X. fastidiosa | Bothrogonia | P. agglomerans |
| | | | ferruginea | |
| Citrus | Citrus variegated | X. fastidiosa | Acrogonia | P. agglomerans |
| | chlorosis | | terminalis | |
| Citrus | Citrus variegated | X. fastidiosa | Dilobopterus | P. agglomerans |
| | chlorosis | | costalimai | |
| Citrus | Citrus variegated | X. fastidiosa | Oncometopia | P. agglomerans |
| | chlorosis | | fascialis | |
| Citrus | Citrus variegated | X. fastidiosa | Sonesimia grossa | P. agglomerans |
| | chlorosis | | | |
| Citrus | Citrus variegated | X. fastidiosa | Hortensia similis | P. agglomerans |
| | chlorosis | | | |
| Citrus | Citrus variegated | X. fastidiosa | Ferrariana sp. | P. agglomerans |
| | chlorosis | | | |
| Citrus | Citrus variegated | X. fastidiosa | Molomea sp. | P. agglomerans |
| | chlorosis | | | |
| Olive | Olive quick decline | X. fastidiosa | Philaenus | P. agglomerans |
| | syndrome | | spumarius | |
| Olive | Olive quick decline | X. fastidiosa | Neophilaenus | P. agglomerans |
| | syndrome | | campestris | |
| Olive | Olive quick decline | X. fastidiosa | Euscelis lineolatus | P. agglomerans |
| | syndrome | | | |

As shown in Table 1, the genetically-modified symbiont microbe can be, or be derived from, Pantoea agglomerans. As used herein, the term “derived from” refers to a microbe that may be genetically-modified from wild-type. Thus, in the context of the present disclosure, a genetically-modified microbe derived from, for example, P. agglomerans allows for genetically modifying any non-wild-type version of the microbe.

While described herein in the context of exemplary embodiments in which the symbiont is genetically modified to produce anti-microbial peptide molecules melittin or SLM to kill the pathogen, the symbiont can be genetically modified to produce any polypeptide to which the pathogen is more sensitive than the symbiont. Thus, in other embodiments, the symbiont may be genetically modified to produce alternative anti-microbial peptide molecules such as, defensins, cecropins, maganins, apidaecin and other related anti-microbial peptides, and engineered antibodies such as V_{H}-V_{L}, V_{H}-V_{H}, V_{H}-fluorophore-V_{L} that recognize any pathogen (e,g, X. fastidiosa) surface protein. In other embodiments, the symbiont may be genetically-modified to produce an antibody or an antibody fragment that specifically binds to the pathogen. The symbiont can be genetically modified to produce, for example, a single chain antibody that binds to surface molecules of X. fastidiosa (Azizi et al., 2012, Appl. Environ. Microbial. 78(8):2638-2647) that can be delivered via P. agglomerans. In still other embodiments, the symbiont can be genetically modified to produce a chimera or fusion of active molecules such as, for example, an antibody:AMP chimera. These chimeras in association with tandem use of anti-microbial peptide secreting strains can increase the potency and delay the development of resistance.

Transforming bacteria to produce different effector molecules and releasing them in the field is easier than producing transgenic insects to manage disease transmission, which can enhance the potential to block disease transmission and/or the ability to manage resistance. Transformed strains of P. agglomerans did not show any growth advantage over wild-type P. agglomerans. Moreover, certain symbionts such as, for example, P. agglomerans tend to lose foreign plasmids over time and revert to wild-type. Thus, the system can be designed to use a symbiont that can alleviate concerns of environmental agencies regarding development of a genetically-modified organism that will permanently outcompete wild-type organisms in a field population. Such systems may therefore be specifically designed for a time-limited application. Farmers may therefore use these strains as a pesticide rather than a perpetual method to control disease. For example, one field application of the system may include spraying AMP-producing P. agglomerans at the end of spring or the start of summer, allowing GWSS to acquire the transformed P. agglomerans, which will make the sharpshooters incompetent for acquiring X. fastidiosa and subsequently reduce transmission.

The genetically-modified symbiont may be released using any suitable method. Exemplary methods include painting the plants, spraying, use of microparticles etc. In some embodiments, the genetically-modified symbiont may be released using a microparticle-based strategy (Arora et al., 2015, BMC Biotechnology 15:59), that addresses other concerns related to environmental contamination by transgenes and this strategy can be used in field to deliver transformed P. agglomerans to the sharpshooter gut.

P. agglomerans, especially the biopesticides strains, can be used to deliver foreign molecules not only in the sharpshooters, but also to other vectors in which P. agglomerasn occurs as a commensal bacterium. This bacterium can help to decrease the disease pressure in humans as well as in agriculture setting by targeting different pathogens/parasite inside the vector gut.

In some embodiments, it may be desirable to control the pathogen without resorting to using a genetically-modified symbiont. In such cases, the pathogen may be controlled by recombinantly producing the AMP compound and applying the AMP compound to a crop plant (e.g., grape, citrus, or olive) in an amount effective to inhibit the pathogen after the vector organism acquires the applied AMP compound. In some embodiments, the AMP compound may be prepared with a microparticle (e.g., as described in Example 2, below). The AMP compound, regardless of the delivery formulation, may be applied to the crop plant by, for example, spraying the delivery formulation to the crop plant in the field environment. There, the AMP compound is available for acquisition by the vector organism. When the AMP compound reaches the foregut of the vector organism, it can inhibit the likelihood and extent to which the pathogen can grow in the foregut of the vector organism. In this regard, the AMP compound activity is similar to embodiments in which the AMP compound is produced in the foregut of the vector organism by a microbe genetically modified to produce the AMP compound after being acquired by the vector organism.

Thus, this disclosure describes a composition that includes an antimicrobial peptide formulated for application to a crop plant. In some cases, the formulation can include incorporating the AMP compound into, or affixing or adhering the AMP compound onto, a microparticle. This disclosure also describes a vector organism that includes such a composition in its foregut.

This disclosure further describes methods for controlling a pathogen that is carried by a vector organism and susceptible to an AMP that inhibits growth of the pathogen. Generally, the method includes applying a composition that includes the AMP compound to a crop plant for acquisition by the vector organism, then allowing the vector organism to ingest or otherwise acquire the AMP compound in an amount effective to inhibit growth of the pathogen when the pathogen is exposed to the AMP compound acquired by the vector organism.

As used herein, the term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

Toxicity of AMPs Against P. agglomerans and X. fastidiosa

Overnight culture of P. agglomerans was diluted in 3 mL LB broth to a dilution of 1:100. The bacterial culture was grown at 30° C. in an incubator shaker shaking at 200 rpm until it reached mid-log phase. At mid-log phase the bacteria were diluted to 10⁵-10⁶ colony forming units/mL (CFUs/mL) in LB medium. 90 μL of diluted P. agglomerans was pipetted into sterilized 0.2 mL PCR tubes and to this 10 μL of anti-microbial peptide (melittin or SLM) solution was added. Different anti-microbial peptide concentrations were tested for their toxicity towards Xylella and Pantoea. These tubes were incubated overnight at 30° C. and next morning OD₆₀₀ was determined.

X. fastidiosa strain Temecula was grown in PD3 medium in a shaker incubator at 28° C. and 200 rpm until it reached its log phase. The culture was then taken out and was diluted to a concentration of 10⁵-10⁶ CFUs/mL in PD3 medium. 90 μL of diluted X. fastidiosa was mixed with 10 μL of anti-microbial peptide (melittin or SLM) in a sterilized 0.2 mL PCR tube and was incubated overnight at 28° C. in a shaker incubator. Next day X. fastidiosa was plated on to PD3 agar. These plates were incubated at 28° C. for 10 days and CFUs were counted.

### Plasmid Construction

Melittin sense and antisense sequences with NheI and XmaI overhang were ordered from Integrated DNA Technologies, Inc. (Coralville, Iowa, USA) and were annealed to one another by lowering the temperature by 1° C./min from 95° C. to 50° C.

The coding region of scorpine-like molecule (SLM), an anti-microbial peptide from Vaejovis mexicanus venom, was amplified from a plasmid (kindly provided by Dr. Lorival D. Possani; Quintero-Hernandez et al., 2015, PloS ONE 10(2), e0117188. doi:10.1371/journal.pone.0117188) using forward primer (ScoHlyAF1.1) CAGCTAGCGGTTGGATAAGCGAG (SEQ ID NO:2); and reverse Primer (ScoHlyAR1.1) TTTTTTATAGGCACGGGGTATACC (SEQ ID NO:3). The product was cut using restriction enzymes NheI and SmaI.

The plasmid pEHLYA2-SD (kindly provided by Dr. Luis A. Fernandez; Fernandez et al., 2000, Appl Environ Microbiol 66(11): 5024-5029), containing hlyA secretion signal of E. coli hemolysin secretion system with an in-frame E-tag at 5′ end of the secretion signal, also was cut using restriction enzymes NheI and SmaI/XmaI. Melittin, and SLM genes were ligated into linearized pEHLYA2-SD plasmid. The in-frame presence of both melittin and SLM genes was confirmed by sequencing.

P. agglomerans Transformation

Overnight P. agglomerans culture was diluted in LB broth and grown to an OD₆₀₀ of 0.6-0.7 (mid-log phase). These cells were centrifuged at 4° C. and 8000 rpm for 10 minutes and supernatant was removed. The cells were washed with ice cold sterilized water. The final cell pellet of competent cells was re-suspended in 1 mL 10% glycerol. 80 μL of competent cell suspension was aliquoted into microcentrifuge tubes. 1 μL of pVDL9.3 plasmid, containing HlyB, HlyD and chloramphenicol resistance genes, was added to 80 μL of competent cells and transferred to ice cold 1 mm cuvette. These cells were electroporated at 2.0 kv, 25 microF. The cells were then plated onto chloramphenicol-containing LB agar overnight. The next morning, the colonies were selected and presence of plasmid was confirmed.

pVDL9.3 plasmid-containing P. agglomerans cells were made competent using the above-described protocol and were transformed with pEHLYA2-SD plasmid having different anti-microbial peptide coding regions. P. agglomerans containing both pVDL9.3 and pEHLYA2-SD plasmids were selected on LB agar containing carbenicillin and chloramphenicol.

### Detection of Melittin and SLM in Spent Medium

Overnight cultures of P. agglomerans were centrifuged at 10,000 rpm and the supernatants were collected. These supernatant from each culture was concentrated using 10 kDa NMWL filter (catalog # MRCPRT010, EMD Billerica, Mass., USA). 20 μL of concentrated spent medium was mixed with 5 μL of loading dye and ran on a polyacrylamide gel at a constant electric potential of 150V. The proteins were then transferred to nitrocellulose membrane at a constant potential difference of 30V. The nitrocellulose membrane was incubated with primary rabbit anti-E-tag antibody, which was diluted to a dilution of 1:1000 in 10% milk-TBST, at room temperature for one hour. This membrane was washed and incubated with mouse anti-rabbit secondary antibody with AP conjugate, diluted in milk-TBST to a dilution to 1:5000, for one hour. The membrane was washed again and was developed using NBT and BCIP.

Presence of melittin in the supernatant was confirmed using rabbit anti-melittin serum (1:1000 in milk-TBST) keeping other conditions same as mentioned above.
X. fastidiosa Transmission Blocking Assays

To demonstrate transmission blocking, field caught sharpshooters were first exposed to modified P. agglomerans that had been painted onto the stems of grape plants. The paratransgenic insects were then transferred to plants infected with X. fastidiosa for acquisition of the pathogen. The ability of the “infected” paratransgenic insects to transmit X. fastidiosa is assessed by placing these insects onto naïve plants. In these experiments, slurries containing 10¹⁰ CFUs of respective P. agglomerans lines were painted onto the grape stem as described (Arora et al., 2015, BMC Biothechnology, 15:59). Sharpshooters were exposed to the P. agglomerans painted plants for 48 hours, prior to transfer to X. fastidiosa infected plants for another 48 hours. Transmission of disease is modeled by placing two “infected” paratransgenic insects on naive grape plants for 24 hours. Following “infection” the insects were removed and surface sterilized. DNA was extracted and presence of X. fastidiosa within the insect head was tested by real-time PCR. The grape plants “infected” by the paratransgenic insects were monitored in a greenhouse for up to 30 weeks. At the end of the testing period, plants were tested for presence of X. fastidiosa by real-time PCR.

DNA Extraction from the Insect Head

The sharpshooters were surface sterilized by washing them in 70% ethanol for two minutes followed by washing in 10% bleach for two minutes. These sharpshooters were then washed twice in sterilized water for two minutes each. After removing from the sterilized sharpshooters' bodies using surgical blade the sharpshooters' heads were homogenized in 200 PBS and DNA was extracted using DNeasy Blood and Tissue Kit (Qiagen, Valencia, Calif.) following manufacturer's instructions.

DNA Extraction from Plant Tissues

30 weeks after inoculation, stems of approximately three inches were cut from grape plants. These stems were surface sterilized by washing in 70% ethanol and 10% bleach for two minutes each, followed by 2× washing in sterilized water for two minutes. These stems were put in Adagia bags and were homogenized in 800 μL of lytic buffer (20 mM Tris-Cl pH 8.0, 70 mM sodium EDTA, 2 mM NaCl, 20 mM sodium metabisulfite, Na₂S₂O₅) using mortar pestle. 200 of homogenized solution was pipetted into a 1.5 mL microcentrifuge tube. To this, 40 μL of 5% sodium sarkosyl and 1.5 μL of proteinase K were added and incubated at 55° C. for one hour. This solution was then centrifuged at 13,000 rpm for 15 minutes and DNA was purified from supernatant using GeneClean kit (Catalog #111001200, MP Biomedicals, Santa Ana, Calif.) following manufacturer's instructions.

### Real-Time PCR

Real time-PCR was performed using ITS-specific primers and probes previously described (Schaad et al., 2002, Phytopathology 92(7):721-728). The 20 μL reaction was performed in 0.1 mL strip tubes containing 10 μL 2×IQ Supermix (Bio-Rad Laboratories, Inc., Hercules, Calif.), 100 nM forward primer, 200 nM reverse primer, 200 nM TAQMAN probe (Applied Biosystems, Thermo Fisher Scentific, Inc., Waltham, Mass.) with FAM fluorophore, 5.8 of PCR-grade water and 2 μL of template DNA. The real-time PCR was performed on the Eppendorf Realplex machine (Thermo Fisher Scentific, Inc., Waltham, Mass.). The enzyme activation step was performed at 95° C. for three minutes followed by denaturation at 95° C. for 15 seconds, and annealing and extension at 58° C. for one minute. The PCR was run for 40 cycles.

### Detecting Accumulated of AMPs Inside the Insect Body

The glassy-winged sharpshooters were surface sterilized as mentioned above. The whole sharpshooters were then homogenized in PBS. The homogenized solution was centrifuged at 13,000 rpm for 10 minutes and supernatant was used for anti-microbial peptide detection. 20 of supernatant mixed with 5 μL of reducing marker was run on a polyacrylamide gel. The proteins were transferred on to the nitrocellulose membrane as mentioned above and proteins were detected using primary rabbit anti-E-tag antibody as mentioned above.

### Example 2

### Sharpshooter Maintenance

The glassy-winged sharpshooters were collected from crepe myrtle (Lagerstroemia sp.) trees in Riverside, Calif. These sharpshooters were kept on basil plants until they were used.

### Bacterial Strains, Culture Conditions and Painting on to the Plant

Escherichia coli strain XL1-Blue (Agilent Technologies, Inc., Santa Clara, Calif.) was used to maintain plasmids and for gene cloning. Pantoea agglomerans E325, an EPA-approved biological control agent, was used to express and deliver different AMP molecules inside the sharpshooter gut. Both E. coli and P. agglomerans were grown in LB agar or broth. P. agglomerans and E. coli were cultured on agar plates at 30° C. and at 37° C., respectively. Broth cultures were grown at the same temperatures in a shaker incubator (200 rpm). Carbenicillin at a concentration of 100 μg/mL or chloramphenicol at a concentration of 35 μg/mL was added when needed.

X. fastidiosa Temecula strain was used in toxicity assays and was cultured in PD3 agar at 28° C. or in PD3 broth at 28° C. The shaker was agitated at 175-200 rpm to grow X. fastidiosa in broth culture.

MIC of MBC AMPs Against P. agglomerans and X. fastidiosa

P. agglomerans was grown in LB broth overnight at 200 rpm in a shaker incubator at 30° C. Next morning P. agglomerans was diluted 1/100 in 3 mL LB broth and grown at 30° C. to mid log phase. At mid log phase the bacteria were diluted (CFUs/mL) in LB medium to 10⁵-10⁶ colony forming units/mL. 90 μL of diluted P. agglomerans were pipetted into sterilized 0.2 mL PCR tubes and to this 10 μL of 10× test concentration of either melittin or SLM was added. These tubes were incubated overnight at 30° C. and next morning OD₆₀₀ was determined.

X. fastidiosa strain Temecula was grown in PD3 medium in a shaker incubator at 28° C. and 200 rpm till it reached its log phase. The culture was then taken out and diluted to a concentration of 10⁵-10⁶ CFUs/mL in PD3 medium. 90 μL of diluted X. fastidiosa was mixed with 10 μL of 10× final concentration either AMP in a sterilized 0.2 mL PCR tube and was incubated overnight at 28° C. in a shaker incubator. X. fastidiosa is a slow growing bacterium, which makes measuring change in OD₆₀₀ of overnight cultures unfeasible. Hence, after overnight treatment with AMPs X. fastidiosa was plated on to PD₃ agar to determine MBC of AMPs against X. fastidiosa. These plates were incubated at 28° C. for 10 days and CFUs were counted.

### Plasmid Construction

Melittin sense and antisense sequences with NheI and XmaI overhang were ordered from Integrated DNA Technologies, Inc. (Coralville, Iowa, USA) and were annealed to themselves by lowering the temperature by 1° C./min from 95° C. to 50° C.

Scorpine like molecule (SLM, an AMP from Vaejovis mexicanus venom) coding region was amplified from a plasmid (kindly provided by Dr. Lourival D. Possani; Quintero-Hernandez et al., 2015, PloS ONE 10(2), e0117188. doi:10.1371/journal.pone.0117188) using forward primer (ScoHlyAF1.1) CAGCTAGCGGTTGGATAAGCGAG (SEQ ID NO:2) and reverse Primer (ScoHlyAR1.1) TTTTTTATAGGCACGGGGTATACC (SEQ ID NO:3). The product was cut using restriction enzymes NheI and SmaI.

The plasmid pEHLYA2-SD (kindly provided by Dr. Luis A. Fernandez (Fernandez et al., 2000, Appl Environ Microbiol 66(11): 5024-5029)—having the hlyA secretion signal of the E. coli hemolysin secretion system—was also cut using restriction enzymes NheI and SmaI. Melittin coding region or SLM coding region was ligated into the linearized pEHLYA2-SD plasmid. The in-frame presence of melittin or SLM coding region was confirmed by sequencing. The successful, in-frame insertion of melittin or SLM coding region resulted in plasmid pEHLYA2-SD-Mel or pEHLYA2-SD-SLM.

P. agglomerans Transformation

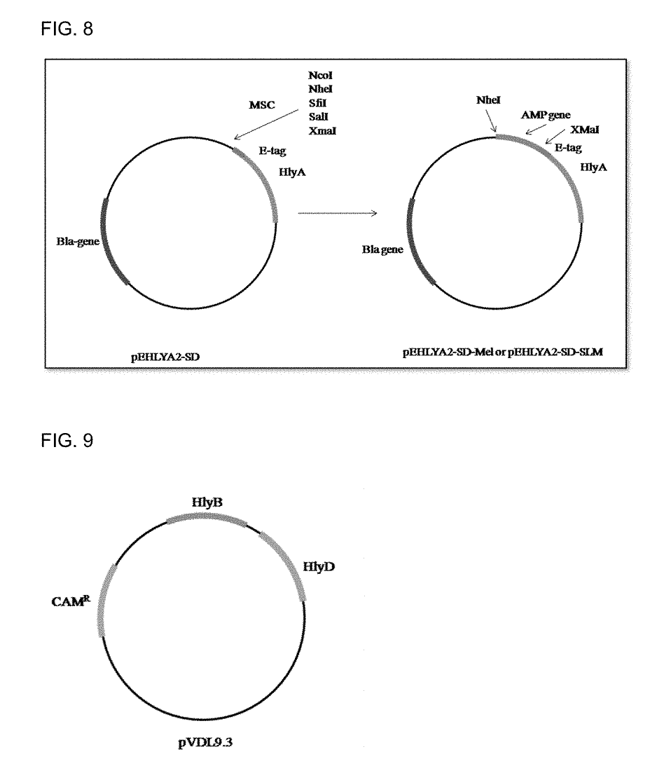

P. agglomerans was diluted in LB broth and grown to an OD₆₀₀ of 0.6-0.7 (mid log phase). These cells were centrifuged at 4° C. and 8000 rpm for 10 minutes and supernatant was removed. The cells were washed with ice cold autoclaved water. The final cell pellet of competent cells was resuspended in 1 mL 10% glycerol. 80 μL of competent cell suspension was aliquoted into microcentrifuge tubes. 1 μL of pVDL9.3 plasmid (FIG. 9) was added to 80 μL of competent cells and transferred to an ice-cold 1 mm cuvette. These cells were electroporated at 2.0 kv, 25 microF. The cells were then plated onto chloramphenicol-containing LB agar. Next morning, the colonies were selected and the presence of plasmid was confirmed.

pVDL9.3 plasmid-containing P. agglomerans cells were made competent using the above-mentioned protocol and were transformed with plasmid pEHLYA2-SD or pEHLYA2-SD-Mel or pEHLYA2-SD-SLM. Transformed P. agglomerans were selected on LB agar containing carbenicillin and chloramphenicol.

P. agglomerans Growth Curve

Overnight cultures of wild-type P. agglomerans, P. agglomerans secreting melittin, P. agglomerans secreting SLM, and P. agglomerans secreting only the HlyA secretion signal were grown at 30° C. in LB broth. Carbenicillin and chloramphenicol were added to LB broth where needed. Next morning, each culture was diluted 1/50 in LB broth without antibiotic. The cultures were grown in a shaker incubator at 30° C. and 200 rpm and OD₆₀₀ was measured every hour.

### Detection of Melittin and SLM in Spent Medium

Overnight cultures of P. agglomerans were centrifuged at 10,000 rpm and the supernatants were collected. The supernatant from each culture was concentrated using 10 kDa NMWL filter (catalog #MRCPRT010, EMD Millpore, Temecula, Calif.). 20 μL of concentrated spent medium was mixed with 5 μL of loading dye and run on a 8-16% precast polyacrylamide gel (Catalog #456-1103, Bio-Rad Laboratories, Inc., Hercules, Calif.) at a constant electric potential of 150V. The proteins were then transferred to a nitrocellulose membrane. The nitrocellulose membrane was first incubated with primary rabbit anti-E-tag antibody, which was diluted to a dilution of 1:1000 in 10% milk-TBST, at room temperature. This membrane was washed five times with TBST and incubated with mouse anti-rabbit antibody with AP conjugate, which was diluted in milk-TBST to a dilution to 1:5000. This membrane was washed five times with TBST and was developed using NBT and BCIP.

Presence of melittin in the supernatant was reconfirmed using rabbit anti-melittin serum using the protocol as mentioned above.

X. fastidiosa Transmission Blocking Assays

P. agglomerans lines were cultured in LB broth and overnight cultures were washed twice with PBS. After washing, 10¹⁰ CFUs of P. agglomerans lines were suspended in 3 mL PBS. Each suspension was mixed with 20 mL 3% guar gum (w/v). 1 mL glycerol and 500 India Ink were added to it before this slurry was painted on to grape stems. The plants were kept overnight to let the guar gum dry. These stems were then covered with sleeve cages and field-collected sharpshooters were released on these plants. The sharpshooters were kept on these plants for 48 hours before putting them on X. fastidiosa-infected plants for another 48 hours. After acquisition access of 48 hours on X. fastidiosa-infected plants, the sharpshooters were collected and two of each sharpshooters were confined on naive grape plants for 24 hours. Two sharpshooters were used to inoculate X. fastidiosa on each naive grape plant to increase the percent transmission, which is usually around 20 percent. The insects were removed after 24 hours, surface sterilized, and DNA was extracted before running real-time PCR. The inoculated grape plants were kept in the greenhouse for 30 weeks and were tested for X. fastidiosa infection via real-time PCR.

DNA Extraction from the Insect Head

The sharpshooters were surface sterilized by washing them in 70% ethanol for two minutes followed by washing in 10% bleach for two minutes. Subsequently, these sharpshooters were washed twice in sterilized water for two minutes. The heads were removed from the sterilized sharpshooters' bodies using surgical blade. The sharpshooter heads were then homogenized in 200 μL PBS using a Kontes homogenizer and DNA was extracted using DNeasy Blood and Tissue Kit (Catalog #69504, Qiagen, Valencia, Calif.) following manufacturer's instructions.

DNA Extraction from Plant Tissues

After 30 weeks of inoculation stems of approximately 10 cm were cut from plants. These stems were sterilized by washing in 70% ethanol and 10% bleach for two minutes each, followed by 2× washing in sterilized water for two minutes. These stems were put in a mesh bag (Agdia Inc., Catalog # ACC 00930/0100, Elkhart, Ind.) and homogenized in 800 μL of lytic buffer (20 mM Tris-Cl pH 8.0, 70 mM sodium EDTA, 2 mM NaCl, 20 mM sodium metabisulfite) using mortar and pestle. 200 μL of plant tissue suspension in lytic buffer was placed in 1.5 mL microcentrifuge tube. This suspension was incubated at 55° C. for one hour after adding 40 μL of 5% sodium sarkosyl and 1.5 μL of proteinase K. After one hour of incubation, this suspension was centrifuged at 13,000 rpm for 15 minutes and supernatant was collected. DNA was purified from the supernatant using a GeneClean kit (Catalog #111001200, MP Biomedicals, Santa Ana, Calif.) following manufacturer's instructions.

### Real-Time PCR

ITS-specific primers and probes described in Schaad et al., 2002, Phytopathology 92(7):721-728) were used to run real time-PCR. The 20 μL reaction was performed in 0.1 mL strip tubes containing 10 μL 2×IQ Supermix (Bio-Rad Laboratories, Inc., Hercules, Calif.), 100 nM forward primer, 200 nM reverse primer, 200 nM TAQMAN probe (Applied Biosystems, Thermo Fisher Scentific, Inc., Waltham, Mass.) with dye, 5.8 μL of PCR-grade water and 2 μL of template DNA. The real-time PCR was performed on the Eppendorf Realplex (Thermo Fisher Scentific, Inc., Waltham, Mass.) at 95° C. for three minutes for enzyme activation followed by denaturation at 95° C. for 15 seconds, and extension and annealing at 62° C. for one minute. The PCR was run for 40 cycles.

### Detecting Accumulation of AMPs Inside the Insect Body

The glassy-winged sharpshooters were surface sterilized as described above. The whole sharpshooters were then homogenized in PBS using a Kontes homogenizer. The homogenized solution was then centrifuged at 13,000 rpm for 10 minutes and supernatant was used for AMP detection. 20 μL of supernatant was mixed with 5 μL of reducing marker and was run on precast Mini PROTEAN TGX gels. Proteins were transferred on to nitrocellulose membranes as mentioned above and accumulation of protein was detected using primary rabbit anti-E-tag antibody as mentioned above.

Accumulation of melittin inside the insect body was confirmed using rabbit anti-melittin serum. The protocol for Western blot was as described above.

### Microencapsulating the AMP

Protonal LF10-60 alginate (FMC BioPolymer, FMC Corp., Philadelphia, Pa.) is prepared at 1-3% (w/v) concentration in de-ionized water and autoclaved prior to use. Purified/synthetic melittin or SLM is mixed with prepared concentrations of alginate and 1% India ink is added to this slurry. The resulting mixture is atomized from an alcohol-sterilized airbrush into a vat containing sterile 0.05 M CaCl₂ with constant agitation from a distance of 20 cm. The resulting microparticles are allowed to harden for 45 minutes. The microparticles are harvested from the mesh and stored for future use.

### Blocking Disease Transmission Application of AMPs-Containing Microparticles

The above prepared microparticles are either sprayed on grape plants after mixing with a surfactant (e.g., SILWET L-77; Momentive Performance Materials, Inc., Columbus, Ohio) or painted on grape plants after mixing with guar gum. The sharpshooters carrying X. fastidiosa in their foregut are allowed to feed on AMPs sprayed/painted plants for 48 hours. Subsequently, these sharpshooters are released on naive plants for 48 hours. After removing from the naive grape plants, the sharpshooters are tested for presence of X. fasitdiosa via real-time PCR as described above. The naive grape plants are kept in greenhouse for 6-8 weeks and are evaluated for PD symptoms. The naive grape plants are also tested for the presence of X. fastidiosa via real time PCR to confirm PD symptoms are due X. fastidiosa infections.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A genetically-modified microbe that is a symbiont of an animal that is a vector organism for a pathogenic microbe, the genetically-modified microbe comprising:
a heterologous polynucleotide that encodes a heterologous polypeptide that reduces transmission of the pathogenic microbe.

2. The genetically-modified microbe of claim 1 derived from Pantoea agglomerans.

3. The genetically-modified microbe of claim 1 wherein the heterologous polypeptide comprises an antimicrobial peptide.

4. The genetically-modified microbe of claim 3 wherein the antimicrobial peptide comprises melittin or scorpine-like molecule (SLM).

5. The genetically-modified microbe of claim 1 wherein the heterologous polypeptide comprises an antibody that specifically binds to the pathogenic microbe or an antibody fragment that specifically binds to the pathogenic microbe.

6. The genetically-modified microbe of claim 1 wherein the heterologous polypeptide comprises an effective portion of an antimicrobial peptide fused to at least a fragment of an antibody that specifically binds to the pathogenic microbe.

7. A paratransgenic organism comprising the genetically-modified microbe of claim 1.

8. A method of reducing transmission of a pathogen between members of a population of host organisms, the method comprising:
applying a composition to a population of host organisms, the composition comprising a heterologous polynucleotide that encodes a heterologous polypeptide that reduces transmission of the pathogen between host organisms in the population; and
allowing vector organisms carrying or at risk of carrying the pathogen to acquire the composition comprising the genetically-modified microbe.

9. A composition comprising:
a microparticle comprising an antimicrobial peptide.

10. The composition of claim 9 wherein the antimicrobial peptide comprises melittin or scorpine-like molecule (SLM).

11. A vector organism comprising:
the composition of claim 10 in its foregut.

12. A method comprising:
applying a composition comprising a microparticle comprising an antimicrobial peptide to a crop plant susceptible to infection by a pathogen that is carried by a vector organism;
allowing the vector organism to acquire the microparticles such that the antimicrobial peptide can inhibit the pathogen.

13. The method of claim 12 wherein the crop plant comprises a member of the genus Vitis or a multispecies hybrid thereof.

14. The method of claim 12 wherein the crop plant comprises a member of the genus Olea or a multispecies hybrid thereof.
